(19) United States
(12) Patent Application Publication
(10) Pub. No.: US 2004/0265235 A1
(43) Pub. Date: Dec. 30, 2004

(54) Magnetic resonance contrast-enhancing agents and method for detecting and imaging artherosclerotic plaque

(76) Inventors: Egidijus Edward Uzgiris, Niskayuna, NY (US); Mohan Mark Amaratunga, Clifton Park, NY (US); William Thomas Dixon, Clifton Park, NY (US); Paritosh Jayant Dhawale, Brookfield, WI (US); Ahmad Nadeem Ishague, Green Island, NY (US)

Correspondence Address: GENERAL ELECTRIC COMPANY, GLOBAL RESEARCH, PATENT DOCKET RM. BLDG. K1-4A59, NISKAYUNA, NY, 12309, US

(21) Appl. No.: 10/609,269

(22) Filed: Jun. 26, 2003

(51) Int. Cl. A61K 49/00

(52) U.S. Cl. 424/9.322

(57) ABSTRACT

Magnetic resonance (“MR”) contrast-enhancing agents comprise poly(amino acid) backbone chain conjugated to chelating moieties that comprise a plurality of carboxylic acid groups that form coordination complexes with paramagnetic ions. A method for detecting atherosclerotic plaque areas comprises administering at least one of these contrast agents into a subject; and obtaining and comparing MR images of and MR signals from the plaque areas before and after administering the contrast agent. The method can be used to assess an effectiveness of a prescribed course of treatment for atherosclerosis.

### BACKGROUND OF THE INVENTION

The present invention relates to magnetic resonance imaging. In particular, the present invention relates to magnetic resonance contrast-enhancing agents and method for detecting and imaging artherosclerotic plaque using such agents.

Coronary atherosclerosis is by far the most frequent cause of ischemic heart disease, and plaque disruption with superimposed thrombosis is the main cause of acute coronary syndromes of unstable angina, myocardial infarction, and sudden death. Recent research has established that coronary inflammation plays a key role in the development of atherosclerosis. Coronary inflammation is typically accompanied by increased permeability of the endothelial layer of the blood vessel, activation of endothelial cells, and monocyte recruitment. Atherogenesis involves several pathological processes: lipid (especially low-density lipids or LDLs) accumulation in the intima; degeneration of LDL stimulating endothelial cells to display adhesion molecules, which latch onto monocytes and T cells; maturation of monocytes into active macrophages, which ingest modified LDLs and increase in size (known then as foam cells); forming the atheromatous core of the plaque; and formation of a cap of smooth muscle cells and fibrous matrix over the atheromatous core. As atherosclerosis becomes advanced, inflammatory substances secreted by foam cells can dangerously weaken the cap by digesting the fibrous matrix and damaging the smooth muscle cells. If the cap ruptures, tissue factor that is generated by foam cells interacts with clot-promoting elements in the blood, causing a thrombus, or clot, to form. If the clot is big enough, it can halt the flow of blood to the heart, producing a heart attack. Thus, there is a need to provide a noninvasive procedure to detect atherosclerosis and to assess the efficacy of any therapeutic intervention.

In-vivo magnetic resonance imaging (MRI) with low molecular-weight contrast-enhancing agents which comprise single molecules, has been used to study plaques in arterial walls. Such low molecular-weight contrast-enhancing agents have been found to diffuse rapidly into plaques. However, due to their small molecular size, they also tend to be cleared rapidly from the body, requiring the completion of the imaging procedure within a very short time after such agents are administered into the patient. In addition, these low molecular-weight agents deliver only a limited number of contrast-enhancing ions to the region of interest.

Therefore, there is a need to provide MRI contrast-enhancing agents that remain longer in the circulation to provide ample time to migrate across the plaque endothelium, but that also easily diffuse through the endothelial layer on the plaque. In addition, it is very desirable to provide MRI contrast-enhancing agents that deliver to the plaque a substantially larger number of contrast-enhancing ions than the prior-art agents. Moreover, it is also very desirable to provide an MRI method for better detecting and imaging plaque areas of the blood vessels.

### SUMMARY OF THE INVENTION

The present invention provides MRI contrast-enhancing agents and an MRI method for imaging plaque areas of the blood vessels. A contrast-enhancing agent of the present invention comprises an extended poly(amino acid) conjugated to chelator moieties that form coordination complexes with paramagnetic ions.

In one aspect of the present invention, each of the chelator moieties comprises a plurality of carboxylic acid groups.

In still another aspect of the present invention, at least 90 percent of the amino acid residues of the poly(amino acid) are conjugated to the chelator moieties.

The present invention also provides a method for acquiring images of at least a wall area of a blood vessel using MRI, the method providing an assessment of atherosclerotic plaque formation in the wall area. The method comprises: administering into a subject at least an MRI contrast-enhancing agent that comprises an extended poly(amino acid) conjugated to chelator moieties that form coordination complexes with paramagnetic ions; and obtaining at least a magnetic resonance (MR) image of the wall area of the blood vessel. The method further comprises locating an area of the blood vessel wall associated with shortened MRI T₁ proton relaxation time, indicating an increased permeability of a blood-vessel endothelial layer.

In one aspect of the present invention, the method comprises repeating the steps of administering the MRI contrast-enhancing agent and obtaining the at least an MR image, and comparing the images to identify a change in the atherosclerotic plaque.

In another aspect of the present invention, the method further comprises acquiring MR signals of the blood vessel areas, the MR images of which are obtained.

Other features and advantages of the present invention will be apparent from a perusal of the following detailed description of the invention and the accompanying drawings in which the same numerals refer to like elements.

### BRIEF DESCRIPTION OF THE DRAWINGS

FIG. 1 is an illustration of inter-chain and intra-chain attraction of polypeptides.

FIG. 2 is an illustration of a highly conjugated polypeptide of the present invention.

### DETAILED DESCRIPTION OF THE INVENTION

The present invention provides MRI contrast-enhancing agents and an MRI method for imaging plaque areas of blood vessels. A contrast-enhancing agent of the present invention comprises an extended poly(amino acid) conjugated to chelator moieties that form coordination complexes with paramagnetic ions. In the present disclosure, the terms “poly(amino acid)” and “polypeptide” are used interchangeably. The term “contrast-enhancing agent” is sometimes abbreviated to “contrast agent.”

Conformation of the Polymeric Contrast-Enhancing Agent

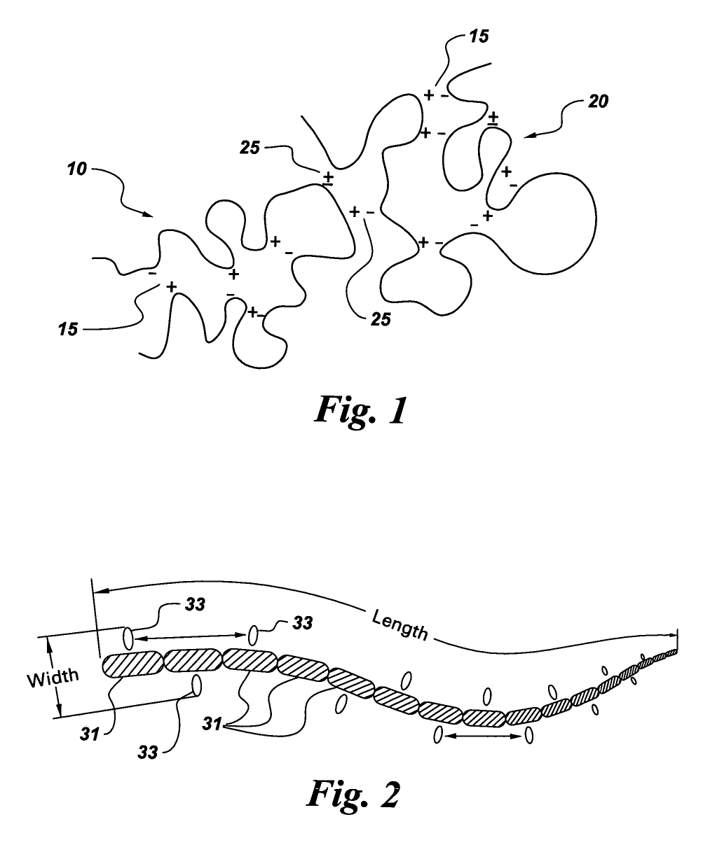

An extended poly(amino acid) contrast-enhancing agent of the present invention has an elongated, worm-like conformation. The conformation of a polymer is a result of interaction of intra-chain charges, which interaction is manifested in the extent of rigidity of the polymer molecule. In general, poly(amino acid) molecules in solution carry opposite charges at the amino and carboxylic acid groups, which interact with each other often to result in a bulky tightly folded or globular conformation. For example, FIG. 1 illustrates two poly(amino acid) chains 10 and 20, each carrying a plurality of positive and negative charges. The segments of the same poly(amino acid) chain 10 or 20 carrying opposite charges attract to each other at 15, resulting in highly folded chains. In addition, opposites charges carried on adjacent chains 10 and 20 also attract to each other at 25, resulting in the formation of large globules, each of which comprises a plurality of chains. On the other hand, a poly(amino acid) chain of the present invention is conjugated, to a large extent, with chelator moieties having net negative charges that inhibit the attraction between segments of the chain so as to result in an elongated conformation. The degree of conjugation of a poly(amino acid) chain of the present invention is at least about 90 percent, preferably at least 95 percent. By “conjugation” or “conjugated,” it is meant in this disclosure that an amino acid residue of the poly(amino acid) chain is attached covalently with at least a portion of another organic molecule, which is the chelator for a cation. Thus, the process of conjugation also includes a process of substitution of at least one atom of an amino acid residue with a portion of the chelator. (The term “residue”, as used in this disclosure, means the remaining portion of a monomeric unit that is linked with portions of other monomeric units to form the polymer.) FIG. 2 illustrates a poly(amino acid) chain comprising amino acid residues 31 linked together through peptide bonds. Each of a very large fraction (greater than 90 percent) of the amino acid residues 31 is conjugated with chelator 33 through a covalent bond. Chelators 33 inhibits, by steric hindrance and charge repulsion, the tendency of the poly(amino acid) to become folded upon itself, resulting a stretched out conformation. Therefore, a contrast-enhancing agent of the present invention can easily enter small pores or spaces, such as a porous space between endothelial cells in an atherosclerotic region of a blood vessel, but at the same time is not easily cleared from the body of the subject. Persistence length is a measure that can quantify the “straightness” of a polymeric chain and is a useful parameter characterizing a contrast-enhancing agent of the present invention. Persistence length is the average projection of the end-to-end distance vector (the vector connecting the two ends of the polymer molecule) on the direction of a selected bond vector. The persistence length can be calculated using the radius of gyration of the polymer molecule, which radius of gyration can be determined by a light scattering experiment. See; e.g., Charles R. Cantor and Paul R. Schimmel, “Biophysical Chemistry, Part III: The Behavior of Biological Macromolecules,” pp. 979-1018, W.H. Freeman and Company, New York, N.Y. (1980); Charles R. Cantor and Paul R. Schimmel, “Biophysical Chemistry, Part II: Techniques for the Study of Biological Structure and Function,” pp. 838-846, W.H. Freeman and Company, New York, N.Y. (1980); and Paul J. Flory, “Statistical Mechanics of Chain Molecules,” pp. 36-38, Oxford University Press, New York, N.Y., 1989. The cited sections of these references are incorporated herein by reference. A contrast-enhancing agent of the present invention has a worm-like shape being essentially a stretched-out, extended chain with little folding. A folded poly(amino acid) with little or no conjugation, has a low persistence length of about 10 angstroms, and is not suitable for use in the present invention. On the other hand, a contrast-enhancing agent of the present invention has a persistence length in the range from about 100 to about 600 angstroms. The back-bone chain of a contrast-enhancing agent of the present invention typically has from about 100 to about 650 monomeric amino acid residues.

The conformation of poly(amino acid) chains is also discussed in U.S. Pat. No. 5,762,909; which is incorporated in its entirety in the present disclosure by reference.

In one approach to produce an effective contrast-enhancing agent complex having a proper persistence length, one eliminates or reduces intra-chain charge interactions as well as restricts rotation about a bond at each peptide link. This may be accomplished by making substitution of the chain with a molecule that provides a steric hindrance, extending as side arms from the main chain.

For example, if the polypeptide backbone chain is poly-L-lysine (“PLL”), which has a positive charge at each lysine, one attaches a sufficient amount of substitutions that would impair peptide bond rotation.

One such method is to attach molecules such as diethylene triamine pentaacetic acid (“DTPA”) at most of the lysine residues. Due to the physical size and the steric hindrance effects of DTPA, there is a physical restraint on peptide bond rotation, which restraint extends the polypeptide into a worm-like configuration. Each of these DTPA molecules is attached at an amine group of a lysine amino acid. The degree of substitution is important in defining the conformation of the overall polypeptide. It was found that substituted PLL works well when it is at least 90 percent substituted with DTPA.

In the case that the polypeptide has both positively and negatively charged sections along its length, such as a polypeptide composed of positively charged amino acids having a low degree of substitution with a negatively charged entity, there is a large degree of folding. However, by further substitution, the charge interactions are reduced, thereby reducing the degree of folding.

T₁ Relaxation Time

When the carrier molecule is in an elongated conformation, the chelator entity, which provides the MR activity, is free to rotate about its attachment point to the main chain, allowing a long T₁ relaxation time of the surrounding water protons, which are the source of the MR signal.

When the carrier molecule is in a globular or highly folded conformation, the paramagnetic ions on the chelator entities are less accessible to the surrounding water protons; and, therefore, a short T₁ relaxation time results.

It was, therefore, found that a high relaxivity (relaxitivity is the inverse of relaxation time) or short relaxation time is associated with a molecule which folds upon itself into a globular conformation, such as albumin, at about 15 sec⁻1 milliMolar⁻¹ (sec⁻¹ mM⁻¹). A low relaxivity or long relaxation time is associated with an elongated molecule such as a highly substituted Gd-DTPA-PLL, in which the Gd can rotate rapidly, having a relaxivity of about 8 sec⁻¹ mM⁻¹.

When the relaxivity of a peptide contrast agent was high, the uptake coefficient of such an agent was invariably low, evidently due to the absence of a reptation movement of the contrast agent molecule, resulting in the exclusion of the contrast agent from narrow passages. Thus, it is important to establish that the peptides being compared for optimum length are all of the same conformation. Relaxivity values of the Gd-PLL for various lengths were tested to be between 7.5 and 10 for average chain length of 92, 219, 455, 633, and 1163 residues, in a 2 Tesla magnet (2 T) at 80 MHz and 23° C. This suggests that a reasonably uniform conformational state was achieved for the peptides being compared.

Charges Carried on the Contrast-Enhancing Agent

Since many in-vivo chemical entities have a negative charge, molecules introduced into the subject must have a net negative charge to reduce agglutination and to allow for stable long circulation in the blood plasma. On the contrary, positively charged molecules tend to stick to cell surfaces, which are generally negatively charged. A high net negative charge is also desirable since it also causes the contrast agent complex molecules to retain their elongated, worm-like conformation.

Preparation of Poly(Amino Acid) Contrast-Enhancing Agents

A wide variety of poly(amino acid) polymers can be used as the backbone chains for synthesis of contrast-enhancing agents of the present invention. The poly(amino acid) can be a homopolymer or a copolymer of at least two types of amino acids. In addition, a wide variety of chelating moieties can be attached to the amino acid residues of the poly(amino acid) backbone chain. The following examples disclose DTPA chelating moiety. However, it should be understood that other polycarboxylic acids that comprise at least a construct of polycarboxylic acid and amine groups can also be used. Such other polycarboxylic acids are disclosed below.

### EXAMPLE 1

### Preparation of Gd-DTPA-PLL Contrast Agent

Under an inert atmosphere, the penta anion of DTPA was prepared by reaction of DTPA (2.97 g, 7.56 mmol) with triethylamine (5.37 ml, 3.9 g, 38.56 mmol) in 35 ml acetonitrile for 50 minutes at 55° C. Isobutylchloroformate (1.10 ml, 1.16 g, 8.47 mmol) was added dropwise to the DTPA penta anion, cooled in an well-equilibrated −45° C. bath, maintained by a Cryotrol temperature controller (Thermo NESLAB, Portsmouth, N.H.). After stirring at this temperature for 1 hour, the resulting thick slurry of the diethylenetriamine tetraaceticacid-isobutyl dianhydride was added dropwise, under ambient atmospheric conditions, to 15 ml of an aqueous 0.1 M NaHCO₃ buffered pH 9 solution of PLL (degree of polymerization (DP)=402, MW=84,000 gmol⁻¹, M_{w}/Mₙ=1.10, 0.25 g, 1.2 mmol lysine residue) at 0° C. (M_{w} is the weight-average molecular weight, and Mₙ is the number-average molecular weight of the polymer.)

After 16 hours of stirring at ambient temperature most (if not all) of the acetonitrile was removed under high vacuum (˜10 microns Hg) over a period of 20 to 25 minutes. A warm water bath was used to maintain uniform temperature, prevent sample bumping, and inhibit vacuum cooling. The resulting solution was centrifuged twice at 5000 rpm and 5° C. to deposit a thick semi-translucent sediment. The supernatant containing the product was purified by dialysis and sometimes further purified by ultrafiltration. The resulting DTPA-polylysine was labeled using hydrated gadolinium citrate at lower pH, such as pH less than 7, preferably less than 6, and more preferably less than 5. Other gadolinium salts, such as gadolinium chloride or gadolinium acetate are also suitable. The efficacy of conjugation was determined by a colorimetric test for the identification of underivatized polylysine amine. Polymer purity was determined by HPLC. Typical values for conjugation ranged from 92-98%. Typical polymer yields raged from 40-60%.

All glassware used in the preparation of the dianhydride was dried by heating under a nitrogen atmosphere. Acetonitrile was distilled from calcium hydride and stored over 4-angstrom molecular sieves. High purity triethylamine and isobutylchloroformate were employed and were stored under inert atmosphere. The dianhydride was prepared in a Morton flask using a mechanical overhead stirrer for achieving high mixing efficiency. Finally, the synthesis up to the DTPA polylysine conjugate was carried out uninterrupted. If necessary, the final polymer can be indefinitely stored at 4° C.

Gd-DTPA-Polylysine Purification

Dialysis:

The polymer solution was loaded into 5 mL regenerated cellulose disposable dialyzers with a molecular weight cut off of 8000 (Sigma-Aldrich catalog number Z36,849-0). The polymer solutions were dialyzed using a Spectra/Por EZ-1 Multidialyzer, against approximately 2 liters of 10 mM NaHCO3 for 24 hours, with constant motion. The buffer was changed after 4-6 hours. The samples were dialyzed for 24 hours. Initial and final dialyzed volumes were noted. The initial and final dialyzed polymer solutions were analyzed by HPLC, without filtering.

Ultrafiltration:

The following devices were used for these experiments: Amicon Centriplus YM-3 centrifugal filter devices, containing a regenerated cellulose membrane with a molecular weight cut off of 3000 (catalog number 4420). The membranes were pre-washed with 50 mM phosphate buffer, pH 7 before use to remove polyethylene glycol. The washing procedure was as follows. Add 14 mL of phosphate buffer to the top of the device. Spin for one hour at 3500 rpm in a Sorvall RC-5B Refrigerated Superspeed Centrifuge refrigerated centrifuge at 10° C. (Dupont Corp., Wilmington Del.). Phosphate buffer from top and bottom of the device was replenished after the washing step. Fresh buffer was added and centrifuged as before. These steps were repeated for a total of four times.

The samples were treated as follows: fourteen mL of polymer solution were added to the top of the device. They were then centrifuged at 3500 rpm for four hours. The filtrate was recovered from the bottom of the device. The retentates were washed by adding 14 mL of 50 mM phosphate buffer, pH 7, and centrifuging for an additional 210 minutes. The filtrate was recovered from the bottom of the device. The retentates were recovered by inverting the top part of the device and centrifuging at 1000 rpm for 2 minutes. All fractions were analyzed by high-pressure liquid chromatography (“HPLC”).

HPLC details:

A Dionex (Sunnyvale, Calif.) DX500 HPLC system equipped with a model PD-40 uv-visible photodiode array detector was used to monitor the synthetic efforts. This system was controlled with Dionex's Peaknet version 5.21 software. For the purposes of this work a Supelco TOSOH Biocep TSK-gel 7.8 mm×30 cm, 10 μM partical size column was utilized. The eluent was 50 mM phosphate buffer, 200 mM NaCl adjusted to pH=7.00 running at 0.6 ml/min with a run time of 35 minutes.

The conjugated polymers produced by the methods described herein can have a degree of conjugation of about 90 percent or higher. A degree of conjugation of 95 percent or higher has been achieved. Such consistently high degrees of conjugation have not been achieved by other prior art processes. The preferred highly conjugated Gd-DTPA-PLL conjugates exhibit superior relaxivity in bulk water (6.8-7.8 1 mol⁻¹ sec⁻¹), as well as penetration in tumor tissues, which also exhibit a high degree of vascular permeability, relative to comparable polymer of lower degrees of conjugation. Such a highly conjugated DTPA-PLL contrast agent has a cross-sectional diameter of about 25 angstroms.

The chemistry and synthesis procedure of Example 1 can be used to prepare contrast agents that comprise, in their backbone chains, residues of amino acids having a free nitrogen-containing group other than lysine, such as histidine, arginine, asparagine, or glutamine. Thus, the poly(amino acid) backbone chain can be polyhistidine, polyarginine, polyasparagine, polyglutamine, or a copolymer of at least two amino acids selected from the group consisting of lysine, histidine, arginine, asparagine, and glutamine.

Specifically, a contrast-enhancing agent of the present invention can comprise a poly(amino acid) selected from the group consisting of polyhistidine, polyarginine, polyasparagine, polyglutamine, and a copolymer of at least two amino acids selected from the group consisting of lysine, histidine, arginine, asparagine, and glutamine, a large fraction (e.g., greater than about 90 percent, preferably greater than 95 percent) of the amino acid residues being conjugated with chelating moieties which form coordination complexes with paramagnetic ions. The chelating moieties can be DTPA or any of the other chelating moieties disclosed below in the section “Other Chelating Moieties.”

### EXAMPLE 2

### Preparation of DTPA-Conjugated Poly(glutamic acid)

A method of preparing a poly(glutamic acid) carrier molecule highly substituted with DTPA, which sterically hinders significant folding of the poly(glutamic acid) backbone chain, resulting in a contrast-enhancing agent having worm-like conformation is described below.

A mixed anhydride of DTPA was prepared according to the method as described in P. F. Sieving, A. D. Watson, and S. M. Rocklage, Bioconjugate Chem. Vol.1, pp. 65-71, (1990).

A flask was charged with 7 ml. acetonitrile and 2.6 g of DTPA. The solution was warmed to 60° C. under a nitrogen atmosphere. Triethylamine was then added via a syringe. The mixture was stirred until homogeneous. The clear solution was then cooled to −30° C. under a nitrogen atmosphere and then 0.5 ml of isobutyl chloroformate was slowly added to result in the anhydride of DTPA.

The anhydride of DTPA is then reacted overnight with ethylene diamine (in which the diamine is in large excess to the anhydride). Ethylene diamine is a suitable choice, giving in the end a DTPA linkage of the desired length to achieve proper steric hindrance against peptide chain bending. The product is separated from the diamine and from DTPA that is not reacted, by ion exchange chromatography. The product has an amine group on one of the acetic acid arms of the pentaacetic acid structure of the DTPA.

Linking this amine-modified DTPA product to the poly(glutamic acid) is done by a carboxyl coupling method. The carboxy acid groups of the poly(glutamic acid) are activated by a coupling reagent, 1-ethyl-3-(3-dimethylaminopropyl) carbodiimide hydrochloride (“EDC”) (Pierce, Rockford, Ill.). The activated group is then combined with the amine-modified DTPA to produce an amide linkage of the DTPA to the peptide backbone as a sidechain which acts as a steric hindrance straightening the polypeptide backbone. The end product is separated by diafiltration.

The resulting poly(glutamic acid) conjugated with DTPA can be converted to Gd-DTPA-poly(glutamic acid) contrast agent by reacting with a gadolinium salt, such as gadolinium citrate, as is disclosed in Example 1.

The chemistry and synthesis procedure of Example 2 can be used to prepare contrast agents that comprise, in their backbone chains, residues of amino acids having a free carboxylic acid group other than glutamic acid, such as aspartic acid. Thus, the poly(amino acid) backbone chain can be poly(aspartic acid) or a copolymer of glutamic acid and aspartic acid.

Specifically, a contrast-enhancing agent of the present invention can comprise a poly(amino acid) selected from the group consisting poly(glutamic acid), poly(aspartic acid), or a copolymer of glutamic acid and aspartic acid; a large fraction (e.g., greater than about 90 percent, preferably greater than 95 percent) of the amino acid residues being conjugated with chelating moieties which form coordination complexes with paramagnetic ions. The chelating moieties can be DTPA or any of the other chelating moieties disclosed below in the section “Other Chelating Moieties.”

In addition, a copolymer of monomeric amino acid residues, each having a free amino group or a free carboxylic acid group, such as a copolymer of lysine and glutamic acid can be used as the backbone chain to prepare a contrast-enhancing agent of the present invention. In this case, an amine-modifed DTPA, such as that prepared according to the procedure of Example 1, would be used to create the chelating moieties extending from the copolymer backbone chain. Such a copolymer is a copolymer of at least a first amino acid selected from the group consisting of lysine, histidine, arginine, asparagine, and glutamine; and at least a second amino acid selected from the group consisting of glutamic acid and aspartic acid.

In one embodiment of the present invention, the poly(amino acid) is a copolymer of lysine and at least one of glutamic acid and aspartic acid.

In another embodiment, a contrast-enhancing agent of the present invention can comprise a poly(amino acid) selected from the group consisting copolymers of at least a first amino acid selected from the group consisting of lysine, histidine, arginine, asparagine, and glutamine; and at least a second amino acid selected from the group consisting of glutamic acid and aspartic acid; a large fraction (e.g., greater than about 90 percent, preferably greater than 95 percent) of the amino acid residues being conjugated with chelating moieties which form coordination complexes with paramagnetic ions. The chelating moieties can be DTPA or any of the other chelating moieties disclosed below in the section “Other Chelating Moieties.”

Other Chelating Moieties

Even though the procedure is illustrated with DTPA, other chelators may also be employed that are capable of being attached to the specific polypeptide being used and that possess a plurality of carboxylic acid groups, which are capable of forming complexes with paramagnetic ions. Non-limiting examples of such other chelators are 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane dietylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

Paramagnetic Ions

Ions that have a partly filled inner shell are suitable to be used in conjunction with a chelator-substituted poly(amino acid) disclosed herein for MRI contrast enhancement. For example, suitable ions are those of transition metal elements, rare earth metals, and actinide elements. Preferred paramagnetic ions are Gd³⁺, Dy³⁺, or a mixture thereof.

Atherosclerotic Plaque Imaging

The present invention provides a method of detecting and imaging atherosclerotic plaque area using an MRI contrast-enhancing agent disclosed herein. The method of the present invention also allows for assessing an effectiveness of a prescribed regimen for treating atherosclerosis. For example, atherosclerotic plaque areas of the blood vessels are imaged using MRI contrast-enhancing agents disclosed herein at time intervals during the course of treatment, and the MR images are compared. Subtle changes in the images are detected more readily due to an increased contrast brought about by the ability of a contrast agent of the present invention to penetrate the areas of the endothelial layer on the plaques.

The method for detecting an atherosclerotic plaque area comprises: (a) administering into a subject a predetermined dose of at least an MRI contrast-enhancing agent that comprises an extended poly(amino acid) conjugated to chelator moieties that form coordination complexes with paramagnetic ions; and (b) obtaining MR images of and acquiring MR signals coming from the subject's blood-vessel wall area surrounding a suspected plaque before and after administering the MRI contrast-enhancing agent into the subject. When an atherosclerotic plaque is present, the MR image acquired after the contrast agent has been administered into the subject shows an increased contrast and an increased MR signal compared to the image and signal acquired before administering the contrast-enhancing agent. Such an increased contrast and increased MR signal are a result of an increase in MR T₁ relaxation time. For example, an increase in the MR signal of 10 percent or more can signify the presence of an atherosclerotic plaque in the area under investigation.

In one aspect of the method, the MR contrast-enhancing agent is administered into the subject at a dose in the range from about 0.01 to about 0.05 moles Gd/kg of body weight of the subject. MR images and signals are acquired within 48 hours after the MR contrast agent is first administered into the subject. An MRI system that can be used for practicing a method of the present invention is disclosed in U.S. Pat. No. 6,235,264; which is incorporated herein by reference in its entirety. In one aspect of the present invention, a contrast-enhancing agent is administered intravenously into a subject. A contrast-enhancing agent can also be administered orally under appropriate circumstances.

In another aspect of the present invention, a method for assessing an effectiveness of a prescribed regimen for treating atheroslerosis comprises: (a) obtaining at least a base-line MR image of acquiring a base-line MR signal from a subject's blood-vessel wall area surrounding a suspected atherosclerotic plaque; (b) administering a first time into a subject a predetermined dose of at least an MRI contrast-enhancing agent that comprises an extended poly(amino acid) conjugated to chelator moieties that form coordination complexes with paramagnetic ions; (c) obtaining pre-treatment MR images of and acquiring pre-treatment MR signals coming from the subject's blood-vessel wall area surrounding the suspected atherosclerotic plaque after administering the predetermined dose of the MRI contrast-enhancing agent into the subject; (d) treating an atherosclerotic condition the subject with the prescribed regimen; (e) administering a second time into the subject the predetermined dose of said at least an MRI contrast-enhancing agent; (f) obtaining post-treatment MR images of and acquiring post-treatment MR signals coming from the same blood-vessel wall area as in step (c); and (g) comparing post-treatment MR images and post-treatment MR signals to pre-treatment MR images and pre-treatment MR signals to assess the effectiveness of the prescribed regimen. A decrease in MR image contrast or MR signals during the course of the prescribed regimen indicates that the treatment has provided benefit. Such a decrease in MR image contrast or MR signal is a result of a decrease in the MR T₁ relaxation time. The method further comprises repeating steps (e), (f), and (g) at predetermined time intervals during the course of treatment for atherosclerosis.

In one aspect of the method, the MR contrast-enhancing agent is administered into the subject at a dose in the range from about 0.01 to about 0.5 moles Gd/kg of body weight of the subject. MR images and signals are acquired within 48 hours after the dose of MR contrast agent is administered into the subject.

The prescribed regimen for treating atherosclerosis can be, for example, at least one of practicing a prescribed diet and exercise program, taking medication for treating a source of plaque deposit, or a combination thereof.

While various embodiments are described herein, it will be appreciated from the specification that various combinations of elements, variations, equivalents, or improvements therein may be made by those skilled in the art, and are still within the scope of the invention as defined in the appended claims.

What is claimed is:

1. A method for detecting an atherosclerotic plaque area of a subject, the method comprising:
administering into the subject a predetermined dose of at least an MRI contrast-enhancing agent that comprises an extended poly(amino acid) conjugated to chelator moieties that form coordination complexes with paramagnetic ions;
obtaining MR images of and acquiring MR signals coming from the subject's blood-vessel wall area surrounding a suspected plaque before and after the step of administering the MRI contrast-enhancing agent into the subject; and
comparing the MR images and MR signals obtained before the step of administering to the MR images and MR signals obtained after the step of administering to identify the blood vessel wall area having an increased MR image contrast and an increased MR signals, which indicate a presence of atherosclerotic plaque.

2. The method according to claim 1, wherein the poly(amino acid) is selected from the group consisting of homopolymers and copolymers of amino acid residues.

3. The method according to claim 1, wherein the poly(amino acid) is poly-L-lysine.

4. The method according to claim 1, wherein the poly(amino acid) is poly(glutamic acid).

5. The method according to claim 1, wherein the poly(amino acid) comprises a number of amino acid residues in a range from about 100 to about 650.

6. The method according to claim 1, wherein the poly(amino acid) has a persistence length in a range from about 100 to about 600 angstroms.

7. The method according to claim 1, wherein the poly(amino acid) is selected from the group consisting of polyhistidine, polyarginine, polyasparagine, polyglutamine, and copolymers of at least two types of amino acids selected from the group consisting of lysine, histidine, arginine, asparagine, and glutamine.

8. The method according to claim 1, wherein the poly(amino acid) is a copolymer of glutamic acid and aspartic acid.

9. The method according to claim 1, wherein the poly(amino acid) is a copolymer of at least a first type of amino acid selected from the group consisting of lysine, histidine, arginine, asparagine, and glutamine; and at least a second type of amino acid selected from the group consisting of glutamic acid and aspartic acid.

10. The method according to claim 1, wherein the chelator moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

11. The method according to claim 1, wherein the chelator moieties are diethylene triamine pentaacetic acid.

12. The method according to claim 1, wherein at least 90 percent of residues of the poly(amino acid) are conjugated with the chelator moieties.

13. The method according to claim 1, wherein at least 95 percent of residues of the poly(amino acid) are conjugated with the chelator moieties.

14. The method according to claim 12, wherein the chelator moieties are diethylene triamine pentaacetic acid.

15. The method according to claim 1, wherein the paramagnetic ions are selected from the group consisting of ions of transition metal elements, rare earth metal elements, and actinide elements.

16. The method according to claim 1, wherein the paramagnetic ions are selected from the group consisting of Gd³⁺, Dy³⁺, and a mixture thereof.

17. The method according to claim 1, wherein said at least an MRI contrast-enhancing agent is administered into the subject at a dose in a range from about 0.01 to about 0.5 mole Gd/kg od body weight of the subject.

18. The method according to claim 1, wherein the MR images and the MR signals obtained after the step of administering are obtained within 48 hours after said administering.

19. A method for detecting an atherosclerotic plaque area of a subject, the method comprising:
administering into the subject a predetermined dose of at least an MRI contrast-enhancing agent that comprises an extended poly(amino acid) conjugated to chelator moieties that form coordination complexes with paramagnetic ions, wherein a degree of conjugation is at least 90 percent;
obtaining MR images of and acquiring MR signals coming from the subject's blood-vessel wall area surrounding a suspected plaque before and after the step of administering the MRI contrast-enhancing agent into the subject; and
comparing the M images and MR signals obtained before the step of administering to the M images and MR signals obtained after the step of administering to identify the blood vessel wall area having an increased MR image contrast and an increased MR signals, which indicate a presence of atherosclerotic plaque;
wherein the poly(amino acid) is selected from the group consisting of poly-L-lysine, polyhistidine, polyarginine, polyasparagine, polyglutamine, poly(glutamic acid), poly(aspartic acid), and copolymers of at least two types of amino acids selected from the group consisting of lysine, histidine, arginine, asparagine, glutamine, glutamic acid, and aspartic acid; the poly(amino acid) has a number of amino acid residues in a range from about 100 to about 650; the chelator moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof; the MRI contrast-enhancing agent is administered into the subject at a dose in a range from about 0.01 to about 0.5 mole Gd/kg of body weight of the subject.

20. A method for assessing an effectiveness of a prescribed regimen for treating atherosclerosis, the method comprising:
obtaining at least a base-line MR image of and acquiring a base-line MR signal from a subject's blood-vessel wall area surrounding a suspected atherosclerotic plaque;
administering a first time into the subject a predetermined dose of at least an MRI contrast-enhancing agent that comprises an extended poly(amino acid) conjugated to chelator moieties that form coordination complexes with paramagnetic ions;
obtaining pre-treatment MR images of and acquiring pre-treatment MR signals coming from the subject's blood-vessel wall area surrounding the suspected atherosclerotic plaque after administering the predetermined dose of the MRI contrast-enhancing agent into the subject;
treating an atherosclerotic condition of the subject with a prescribed regimen;
administering a second time into the subject the predetermined dose of said at least an MRI contrast-enhancing agent;
obtaining post-treatment MR images of and acquiring post-treatment MR signals coming from the same blood-vessel wall area as in the step of obtaining pre-treatment MR images and acquiring pre-treatment MR signals; and
comparing post-treatment MR images and post-treatment MR signals to pre-treatment MR images and pre-treatment MR signals to assess the effectiveness of the prescribed regimen;
wherein a decrease in a contrast of MR images and in MR signals indicates that the prescribed regiment is effective.

21. The method according to claim 20, wherein the poly(amino acid) is selected from the group consisting of homopolymers and copolymers of amino acid residues.

22. The method according to claim 20, wherein the poly(amino acid) is poly-L-lysine.

23. The method according to claim 20, wherein the poly(amino acid) is poly(glutamic acid).

24. The method according to claim 20, wherein the poly(amino acid) is selected from the group consisting of polyhistidine, polyarginine, polyasparagine, polyglutamine, and copolymers of at least two types of amino acids selected from the group consisting of lysine, histidine, arginine, asparagine, and glutamine.

25. The method according to claim 20, wherein the poly(amino acid) is a copolymer of glutamic acid and aspartic acid.

26. The method according to claim 20, wherein the poly(amino acid) is a copolymer of at least a first type of amino acid selected from the group consisting of lysine, histidine, arginine, asparagine, and glutamine; and at least a second type of amino acid selected from the group consisting of glutamic acid and aspartic acid.

27. The method according to claim 20, wherein the chelator moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′, N″,′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

28. The method according to claim 20, wherein the chelator moieties are diethylene triamine pentaacetic acid.

29. The method according to claim 20, wherein at least 90 percent of residues of the poly(amino acid) are conjugated with the chelator moieties.

30. The method according to claim 20, wherein at least 95 percent of residues of the poly(amino acid) are conjugated with the chelator moieties.

31. The method according to claim 29, wherein the chelator moieties are diethylene triamine pentaacetic acid.

32. The method according to claim 20, wherein the paramagnetic ions are selected from the group consisting of ions of transition metal elements, rare earth metal elements, and actinide elements.

33. The method according to claim 20, wherein the paramagnetic ions are selected from the group consisting of Gd³⁺, Dy³⁺, and a mixture thereof.

34. The method according to claim 20, wherein said at least an MRI contrast-enhancing agent is administered into the subject at a dose in a range from about 0.01 to about 0.05 mole Gd/kg od body weight of the subject.

35. The method according to claim 20, wherein the MR images and the MR signals obtained after the step of administering are obtained within 48 hours after said administering.

36. A method for assessing an effectiveness of a prescribed regimen for treating atherosclerosis, the method comprising:
obtaining at least a base-line MR image of and acquiring a base-line MR signal from a subject's blood-vessel wall area surrounding a suspected atherosclerotic plaque;
administering a first time into the subject a predetermined dose of at least an MRI contrast-enhancing agent that comprises an extended poly(amino acid) conjugated to chelator moieties that form coordination complexes with paramagnetic ions, wherein a degree of conjugation is at least 90 percent;
obtaining pre-treatment MR images of and acquiring pre-treatment MR signals coming from the subject's blood-vessel wall area surrounding the suspected atherosclerotic plaque after administering the predetermined dose of the MRI contrast-enhancing agent into the subject;
treating an atherosclerotic condition of the subject with a prescribed regimen;
administering a second time into the subject the predetermined dose of said at least an MRI contrast-enhancing agent;
obtaining post-treatment MR images of and acquiring post-treatment MR signals coming from the same blood-vessel wall area as in the step of obtaining pre-treatment MR images and acquiring pre-treatment MR signals; and
comparing post-treatment MR images and post-treatment M signals to pre-treatment MR images and pre-treatment MR signals to assess the effectiveness of the prescribed regimen;
wherein a decrease in a contrast of MR images and in MR signals indicates that the prescribed regiment is effective; and wherein the poly(amino acid) is selected from the group consisting of poly-L-lysine, polyhistidine, polyarginine, polyasparagine, polyglutamine, poly(glutamic acid), poly(aspartic acid), and copolymers of at least two types of amino acids selected from the group consisting of lysine, histidine, arginine, asparagine, glutamine, glutamic acid, and aspartic acid; the poly(amino acid) has a number of amino acid residues in a range from about 100 to about 650; the chelator moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof; the MRI contrast-enhancing agent is administered into the subject at a dose in a range from about 0.01 to about 0.5 mole Gd/kg of body weight of the subject.

37. An MRI contrast-enhancing agent comprising a poly(amino acid) conjugated with chelating moieties that form coordination complexes with paramagnetic ions; wherein the poly(amino acid) is selected from the group consisting of polyhistidine, polyarginine, polyasparagine, polyglutamine, and copolymers of at least two types of amino acids that are selected from the group consisting of lysine, histidine, arginine, asparagine, and glutamine; and wherein at least about 90 percent of amino acid residues of the the poly(amino acid) are conjugated with the chelating moieties.

38. The MRI contrast-enhancing agent according to claim 37, wherein the chelating moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

39. The MRI contrast-enhancing agent according to claim 37, wherein the poly(amino acid) comprises a number of amino acid residues in a range from about 100 to about 650.

40. The MRI contrast-enhancing agent according to claim 37, wherein the poly(amino acid) has a persistence length from about 100 to about 600 angstroms.

41. The MRI contrast-enhancing agent according to claim 37, wherein the chelating moieties are diethylene triamine pentaacetic acid.

42. The MRI contrast-enhancing agent according to claim 37, wherein the paramagnetic ions are selected from the group consisting of ions of transition metals, rare earth metals, and actinide elements.

43. The MRI contrast-enhancing agent according to claim 37, wherein the paramagnetic ions are selected from the group consisting of Gd³⁺, Dy³⁺, and a mixture thereof.

44. An MRI contrast-enhancing agent comprising a poly(amino acid) conjugated with chelating moieties that form coordination complexes with paramagnetic ions; wherein the poly(amino acid) is a copolymer of at least a first type of amino acid selected from the group consisting of histidine, arginine, asparagine, and glutamine; and at least a second type of amino acid selected from the group consisting of glutamic acid and aspartic acid; and wherein at least about 90 percent of amino acid residues of the the poly(amino acid) are conjugated with the chelating moieties.

45. An MRI contrast-enhancing agent comprising a poly(amino acid) conjugated with chelating moieties that form coordination complexes with paramagnetic ions; wherein the poly(amino acid) is a copolymer of at least a first type of amino acid selected from the group consisting of lysine, histidine, arginine, asparagine, and glutamine; and a second type of amino acid of aspartic acid; and wherein at least about 90 percent of amino acid residues of the poly(amino acid) are conjugated with the chelating moieties.

46. The MRI contrast-enhancing agent according to claim 44, wherein the chelating moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

47. The MRI contrast-enhancing agent according to claim 44, wherein the poly(amino acid) comprises a number of amino acid residues in a range from about 100 to about 650.

48. The MRI contrast-enhancing agent according to claim 44, wherein the poly(amino acid) has a persistence length from about 100 to about 600 angstroms.

49. The MRI contrast-enhancing agent according to claim 44, wherein the chelating moieties are diethylene triamine pentaacetic acid.

50. The MRI contrast-enhancing agent according to claim 44, wherein the paramagnetic ions are selected from the group consisting of ions of transition metals, rare earth metals, and actinide elements.

51. The MRI contrast-enhancing agent according to claim 44, wherein the paramagnetic ions are selected from the group consisting of Gd³⁺, Dy³⁺, and a mixture thereof.

52. The MRI contrast-enhancing agent according to claim 45, wherein the chelating moieties are selected from the group consisting of diethylene triamine pentaacetic acid; 1,4,7,10-tetraazacyclododecane-N,N′,N″,N′″-tetraacetic acid; p-isothiocyanatobenzyl-1,4,7,10-tetraazacyclododecane-1,4,7,10-tetraacetic acid; 1,4,7,10-tetraazacyclododecane-N,N′,N″-triacetic acid; 1,4,7,10-tetraazacyclododecane-1,4,7,10-tetrakis(2-propionic acid); 3,6,9-triaza-12-oxa-3,6,9-tricarboxymethylene-10-carboxy-13-phenyl-tridecanoic acid; 1,4,7-triazacyclononane-N,N′,N″-triacetic acid; 1,4,8,11-tetraazacyclotetradecane-N,N′,N″,N′″-tetraacetic acid; triethylene tetraamine hexaacetic acid; trans-1,2-diaminohexane tetraacetic acid; 1,4,7,10-tetraazacyclododecane-1-(2-hydroxypropyl)4,7,10-triacetic acid; trans-cyclohexane-diamine tetraacetic acid; trans(1,2)-cyclohexane dietylene triamine pentaacetic acid; 1-oxa-4,7,10-triazacyclododecane-N,N′,N″-triacetic acid; 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

53. The MRI contrast-enhancing agent according to claim 45, wherein the poly(amino acid) comprises a number of amino acid residues in a range from about 100 to about 650.

54. The MRI contrast-enhancing agent according to claim 45, wherein the poly(amino acid) has a persistence length from about 100 to about 600 angstroms.

55. The MRI contrast-enhancing agent according to claim 45, wherein the chelating moieties are diethylene triamine pentaacetic acid.

56. The MRI contrast-enhancing agent according to claim 45, wherein the paramagnetic ions are selected from the group consisting of ions of transition metals, rare earth metals, and actinide elements.

57. The MRI contrast-enhancing agent according to claim 45, wherein the paramagnetic ions are selected from the group consisting of Gd³⁺, Dy³⁺, and a mixture thereof.
